(19) United States
(12) Patent Application Publication
(10) Pub. No.: US 2008/0219592 A1
(43) Pub. Date: Sep. 11, 2008

(54) Self-similar ordered microstructural arrays of amphiphilic molecules

(76) Inventor: Fatemeh Mojtabai, Lexington, MA (US)

Correspondence Address: LAHIVE & COCKFIELD, LLP;FLOOR 30, SUITE 3000, ONE POST OFFICE SQUARE, BOSTON, MA, 02109, US

(21) Appl. No.: 12/004,640

(22) Filed: Dec. 21, 2007

Related U.S. Application Data
(63) Continuation of application No. 10/424,165, filed on Apr. 25, 2003, now abandoned.
(60) Provisional application No. 60/375,899, filed on Apr. 25, 2002.

(51) Int. Cl. G06K 9/20; G01N 33/48

(52) U.S. Cl. 382/285; 702/21

(57) ABSTRACT

The invention pertains, at least in part, to a method for determining the structure of an amphiphilic molecule using Self-Similar Microstructure Arrays.

### RELATED APPLICATIONS

This application is a continuation of U.S. patent application Ser. No. 10/424,165, filed Apr. 25, 2003; which claims priority to U.S. Provisional Patent Application Ser. No. 60/375,899, filed on Apr. 25, 2002. This application is also related to U.S. Ser. No. 10/003,468, filed Oct. 23, 2001 and U.S. Provisional Patent Application Ser. No. 60/242,913, filed on Oct. 24, 2000. The entire contents of each of these applications are hereby incorporated herein by reference in their entirety.

### BACKGROUND OF THE INVENTION

Protein structure information is indispensable for the design of effective drugs. Designers need detailed structures of proteins, to atomic resolution, so that they can tailor their drugs to interact with specific target areas in a protein molecule. Conventionally, x-ray crystallography has been used to elucidate the three-dimensional (3-D) structure of proteins. This technique can accurately identify the location of atoms by diffracting x-rays from innumerable protein molecules stacked up in an ordered form, so called “crystal”. However, crystallographers have yet to overcome the fact that most proteins do not readily form ordered assemblies. Particularly important but extremely difficult to crystallize are membrane proteins. Membrane proteins are not soluble, therefore conventional three-dimensional crystallization techniques usually do not work for them.

To date, the number of transmembrane proteins crystallized remains small. Less than 40 out of the over 20,000 structure holdings in the Protein Data Bank (PDB) are membrane proteins. In a 1998 report of the Committee for the National Magnetic Resonance Collaboration, it was stated that “even though membrane proteins represent 30% of the proteome, relatively little is known about the structure of these proteins, because of their resistance to crystallization.”

In general, membrane proteins are comprised of hydrophobic portions within their transmembrane regions, which render them insoluble in water. Consequently, unlike soluble proteins, membrane proteins do not form the monodispersed, isotropic solutions needed to grow crystals. This accounts for the near absence of structure information for transmembrane portions of most membrane proteins. In contrast, in some cases the soluble extracellular and intracellular domains of membrane proteins which lack the hydrophobic regions, have been successfully crystallized.

### SUMMARY OF THE INVENTION

The invention pertains, at least in part, to industrial-scale identification of membrane protein targets. The invention pertains to a rapid and low-cost approach for fabrication of organized assemblies of amphiphilic molecules, such as membrane proteins, as a way to identify their structure, at nano-scale regimes. The nano-scale experimental constraints are combined with external cutting-edge computational modeling, and visualization software to derive at high-resolution structure of membrane proteins. In certain embodiments, the methods of the invention do not require the use of a highly purified membrane protein preparation.

Furthermore, the methods of the invention provide information on the dynamic conformation of the protein, which is often overlooked in structures obtained by x-ray crystallography. The dynamic conformation of proteins is relevant because it affects their function. The methods of the invention may generate information on dynamic changes of the structure of a membrane protein or a membrane protein complex, which is relevant to signal transduction and molecular recognition events. For example, signal transduction is generally initiated by a change in conformation, dimerization, trimerization of a membrane protein receptor. Signal transduction may also be initiated by heteromerization (e.g. heterodimerization, heterotrimerization etc.) of a membrane receptor with another membrane receptor or soluble protein upon binding to a signal transducing ligand (e.g. through formation of heteromeric complexes as in the TGF β signaling through formation of types I and II serine/threonine kinase receptors) or sometimes in the absence of a ligand.

In one embodiment, the invention pertains to a method for determining the size of an amphiphilic molecule. The method includes obtaining an ordered structure of amphiphilic molecules; calculating the fractal dimensions of the ordered structure; and analyzing the fractal dimensions, such that the size of the amphiphilic molecule is determined.

In another embodiment, the invention also pertains, at least in part, to a method for generating a three dimensional template of an amphiphilic molecule. The method includes obtaining at least two ordered structures of an amphiphilic molecule, obtaining the 2D contour map of the amphiphilic molecule from the ordered structures; and analyzing the 2D contour maps to generate a three dimensional template of the amphiphilic molecule.

In yet another embodiment, the invention includes a method for obtaining a three dimensional structure of a protein. The method includes obtaining at least two ordered structures of the protein, determining the 2D contour maps of the protein from the ordered structures; analyzing the 2D contour maps to obtain a three dimensional template; and fitting the amino acid sequence of the protein into the three dimensional template to obtain a three dimensional structure of the protein.

In another embodiment, the invention pertains to using the ordered structures of membrane proteins or membrane proteins in complex with another compound (e.g. a small molecule drug or another protein (e.g. soluble protein, membrane protein, receptor), peptide, DNA or RNA) to profile the expression of membrane proteins in diseased vs. healthy tissue or bodily fluids.

### DETAILED DESCRIPTION OF THE INVENTION

The symmetries of crystalline structures are generally associated with translations, rotations and reflections. A fourth symmetry also exists, scale invariance. Scale invariance is generally associated with fractals and self-similarity. Two-dimensional growth of nanostructured arrays with fractal characteristics has been widely studied in inorganic systems. A number of theoretical models have been developed to understand the processes underlying the growth of thin films (Barabasi, A. L. et al. (1995). Fractal Concepts in Surface Growth, Cambridge University Press). Formation of self-similar ordered arrays has not been studied very much in organic systems.

Compression of a lipid monolayer on a liquid layer of higher surface tension is known to produce unusual 2D structures with remarkable long-range orientational order (Weis, R. M. et al. (1984) Nature 310: 47-49). In some cases, the 2D contours form fractal patterns with approximately 1.5 Hausdorff fractal dimension (Miller, A., et al. (1986) Physical Review Letters 56(24): 2633-2636). The shape of the fractals can be understood by diffusion-limited aggregation in two-dimensions. Shape transitions in lipid monolayer domains is attributed to a balance between the interface (line tension) energy and the long-range electrostatic repulsion between lipid dipoles (De Koker, R. et al. (1994) J. Phys. Chem. 98(20): 5389-93).

### 1. Structural Determination Using Ordered Structures

High resolution structural information can be obtained by using ordered structures (e.g., SSOMA arrays). The present invention is directed, at least in part, to a method for deriving a quantitative understanding of individual amphiphilic molecules from the morphology of an ordered structure.

In one embodiment, the invention pertains to a method for determining the size of an amphiphilic molecule. The method includes obtaining an ordered structure of amphiphilic molecules; calculating the fractal dimensions of the ordered structure; and analyzing the fractal dimensions, such that the size of the amphiphilic molecule is determined.

The term “ordered structures” includes ordered arrays and self-similar ordered microstructured arrays of amphiphilic molecules. The ordered structures can be analyzed by imaging techniques known in the art. In an embodiment, the ordered structures may be crystalline, and can be analyzed by appropriate imaging or diffraction techniques, e.g., electromagnetic radiation diffraction, to determine the shape or structure of the amphiphilic molecule. The term includes nanostructures and microstructures. In an embodiment, the ordered structure is comprised of a population of similar, advantageously identical, molecules.

The term “ordered structures” also includes large ordered microstructured arrays. It was found that when large ordered microstructured arrays of two different membrane proteins were formed in monolayers made of crude membrane protein preparations, the morphology of both of the arrays bore a striking similarity to the known molecular structure of the proteins. The microstructured arrays exhibited a remarkable invariance under scale transformations sometimes greater than 30,000 fold. The overlap of their morphology between two extreme magnifications suggests an isotropic self-similarity. Therefore, the invention pertains to a method for analyzing the morphology of the ordered structures to determine the structure of the individual amphiphilic molecules making up the ordered structure.

In one embodiment, the ordered structures form fractal arrays which demonstrate scale invariance. In a further embodiment, the fractal dimensions of the ordered arrays are calculated, and used to determine the dimensions of the individual amphiphilic molecules which make up the population. The fractal dimensions can be obtained using methods known in the art.

The invention also pertains to the mathematical relationship between the macroscopic dimensions of the ordered structures (e.g. SSOMAs) and the molecular dimension of the amphiphilic molecules, such as proteins.

The term “amphiphilic molecules” includes proteins, lipids, lipoproteins, steroids, cholesterol, or other molecules or derivatives thereof which can be applied to a interface, compressed, to yield an ordered structure. In certain embodiments, the term amphiphilic molecules do not include lipids.

The term “protein” includes both naturally occurring, mutant, modified, and labeled (e.g., polypeptides. The protein may be comprised of at least one hydrophobic region on its surface. The protein is not water soluble. In a further embodiment, the protein may be a membrane protein, and/or a cellular receptor.

The term “membrane protein” includes proteins which in their native state are associated with lipid membranes (e.g., nuclear membrane, cellular membrane, mitochondrial membranes, liposomal membranes, endoplasmic reticulum membranes, chloroplast membranes, etc.). The term includes transmembrane proteins, and proteins which are partially or fully embedded in membranes in their native state. Examples of membrane proteins include G-protein coupled receptors (GPCRs), signal transduction receptors, orphan receptors, and other cellular receptors.

The term “membrane proteins” include both extrinsic and intrinsic proteins. Extrinsic membrane proteins are generally located entirely outside of the membrane, but are bound to the membrane by weak molecular attractions (such as, for example, ionic, hydrogen, and/or Van der Waals bonds). Intrinsic membrane proteins are, generally, embedded in the membrane. Intrinsic membrane proteins include, but are not limited to proteins which extend from one side of the membrane to the other, i.e., transmembrane proteins. Examples of transmembrane proteins include, ion channels and ion pumps. The term also includes glycoproteins which comprise carbohydrate sugars covalently attached to the protein. A typical mammalian cell may have several hundred distinct types of glycoprotein studding its plasma membrane.

The statistical significance of the correlation between the macroscopic shape of ordered structures (e.g., Self-Similar Ordered Microstructured Arrays (SSOMAs) with known molecular conformation of the protein can be determined. The ordered structures (e.g., SSOMAs) can be imaged using an automated image acquisition protocol, at a predefined trajectory, step size and time-interval. The images can then be stored into sets for analysis. The shape of the ordered structures (e.g., SSOMAs) can be evaluated in reference to the known structure of the model protein, using pattern matching and statistical analysis. Furthermore, this data can also be used to calculate the fractal dimensions which in turn can be used to determine the size of homologous or related amphiphilic molecules. In another embodiment, the invention also pertains, at least in part, to a method for generating a three dimensional template of an amphiphilic molecule. The method includes obtaining at least two ordered structures of an amphiphilic molecule, obtaining the 2D contour map of the amphiphilic molecule from the ordered structures; and analyzing the 2D contour maps to generate a three dimensional template of the amphiphilic molecule.

The 2D contour maps can be obtained by analyzing the ordered structures with imaging techniques, such as those described in U.S. Ser. No. 10/003,468, incorporated herein by reference. The term “contour map” includes 2D projection of a cross section of the protein, which may advantageously describe surface features, as well as the overall shape of the target protein molecule. Results on two model membrane proteins suggest that the contour maps of the invention describe some of the surface features as well as the overall shape of the amphiphilic molecules of interest. The contour maps show some structural features of the protein, including such features as clefts and pores. The contour maps of the invention may be considered to be somewhat analogous to electron density maps obtained from x-ray diffraction at low-resolution.

The term “imaging” or “imaging techniques” includes methods known in the art using any form of electromagnetic radiation, neutrons, including techniques such as absorption, fluorescence, reflectance, diffraction, scattering and includes illumination techniques such as transmission, reflectance, incident-light fluorescence, confocal, evanescent wave (including total internal reflection fluorescence and surface plasmon resonance), near field, multi-photons, interference, polarized light, chemi-luminescence and scanning probe microscopy techniques such as confocal, atomic force and tunneling.

The invention pertains to methods for generating three dimensional structures of a particular amphiphilic molecule. The three dimensional templates may be obtained using ordered structures (e.g., SSOMAs) of amphiphilic molecules to generate 2D contour maps through imaging techniques. A three dimensional template of the amphiphilic molecule can be obtained by using 2D contour maps of the amphiphilic molecule from several different orientations using computer modeling, visualization software, and other techniques known in the art.

The invention pertains, at least in part, to a method of creating a 3D structure of a protein of interest. The method includes using mathematical relationships based on self-similarity concepts of the 2D contour maps to predict a three dimensional template of the protein. Then, using sequence or other information available about the protein, aligning the amino acid sequence with the 3D template to producing a three dimensional structure of the protein. The three dimensional structure can further be refined by generating additional structures corresponding to the alignment of the sequence with the three dimensional template and 2D contour maps and selecting the best structure by using a scoring function guided by the exact molecular geometry of the protein. The structure can also be further refined by adding side chains of the protein and otherwise refining the model to derive high resolution structure information.

The ordered structures of the invention can be imaged using fluorescence or other imaging techniques. The amphiphilic molecules which make up the ordered structures may lay in many different orientations, and thus each image may show specific details for the amphiphilic molecule at a particular orientation. The individual images of the amphiphilic molecules will be digitized or otherwise converted to a computer analyzable format and sorted into groups that appear to share the same orientation. The images can be sorted by computing the Fourier transforms of the images to obtain a three dimensional template or structure. Preferably, 2D contours of the amphiphilic molecules are obtained for at least three different orientations of the molecule.

Routine homology modeling software and protein prediction protocols are generally based on availability of hundreds and thousands of solved structures of homologous proteins. These templates are then reconstructed to serve as an average template for the target protein. However, these protocols are rarely applicable to membrane proteins with only a few solved structures. The 2D contour maps and the three dimensional templates determined from the ordered structures provide a individualized template for the target protein. The 2D contour maps and the three dimensional templates are based on experimental constraints from the particular protein and not averaged structure data from other purportedly related proteins. The structural data generated from the analysis of the ordered structures can be used in many different stages of the modeling process, including the alignment, scoring and as input for the refinement step.

The invention also pertains to customized computational and modeling software which utilize the measurements and structural data from the ordered structures.

In one embodiment, the invention pertains to a kinetic model that simultaneously incorporates deposition, diffusion, and aggregation.

In another embodiment the invention pertains to formation ordered structures associated with the proliferation of instabilities induced by the diffusion-limited aggregation (DLA). Such irreversible aggregation of small particles to form clusters were previously observed in colloids and coagulated aerosols. Often, diffusion of the particles to the surface of the aggregate is the rate limiting step in the process. Similar growth processes happen sometimes in precipitation of a compound from a supersaturated solution, and in crystallization from supercooled melt. These aggregates sometimes form complicated multibranched forms and dendrites. The rules of the DLA model are known (Witten & Sander (1983), Physical Review B, 27: 5686-5697): the aggregate starts with a seed particle at the origin of a lattice. Other particles diffuse from far away until they arrive at one of the lattice sites adjacent to an occupied site, launch and halt.

As a result this model can lead to formation of indefinitely large clusters with complicated shapes. These models can be applied to investigate the underlying processes that govern the growth of ordered structures (e.g. SSOMAs) of membrane proteins or a membrane protein in complex with other molecule(s). Such understanding will lead to an ability to improve the spatial order in the ordered structures (e.g., SSOMAs).

Imaging the shape or molecular outline of a membrane protein or a complex between a membrane protein and a compound (e.g. a small molecule drug or another protein (e.g. soluble protein, membrane protein, receptor), peptide, DNA or RNA) is important to probing biological recognition events. For example, cytokine-driven interactions appear to be the initial event in signal transduction for haemopoietin interferon receptors, receptor kinases, and the tumor necrosis factor (TNF)/nerve growth factor (NGF) receptors. In these systems, receptor multimerization allows information to pass from the extracellular domain to the cytoplasmic environment without a change in the conformation of the receptor.

In some receptors such as those for epidermal growth factor (EGF) and platelet-derived growth factor (PDGF), dimerization of the receptor leads to activation of their intrinsic kinase activity. In yet another family, of receptors, e.g., the G-protein coupled receptors, seven membrane spanning receptors, it is believed that the binding of the agonist to the receptor initiates a change in the conformation of the receptor that is recognized by the associate G protein. In the signal transduction pathway, binding of molecular messengers (such as the human growth factors) to cell receptors (such as tyrosine kinases TKₛ), initiates a series of complex events that generally starts with a change in conformation or dimerization of the protein, Signal transduction is involved in diverse cellular processes, including cell growth, reproduction, and migration. Any aberrations in these processes may result serious diseases such as cancer, diabetes, cardiovascular disease, and inflammation.

In a further embodiment, the invention pertains to using the ordered structures of membrane proteins or membrane proteins in complex with another compound (e.g. a small molecule drug or another protein (e.g. soluble protein, membrane protein, receptor), peptide, DNA or RNA) to profile the expression of membrane proteins in diseased (including overexpressed levels of membrane protein) vs. healthy (including normal levels of membrane protein) tissue or bodily fluids. Overexpression of the membrane protein is profiled in the diseased state by imaging ordered structures (e.g. SSOMAs) using diseased tissue or bodily fluids. In the healthy state the ordered structures are predominantly not present or exist is smaller quantities and with often with different shapes.

### 2. Methods for Forming Ordered Structures

The ordered structures, which can be used in the methods of the invention, can be formed by any method known in the art. In a further embodiment, the ordered structures are formed by planar membrane compression.

The population of amphiphilic molecules may be compressed by any method, such that an ordered structure is formed. In certain embodiments, the appropriate pressure may be achieved by applying the amphiphilic molecules to the interface and achieving the appropriate pressure to form an ordered structure of the invention without compression.

The term “appropriate pressure” includes the amount of pressure necessary for a particular protein to form a desired ordered structure, e.g., two dimensional or three dimensional ordered structure. In three dimensions, crystallization is promoted in a super-saturated solution. In two-dimensions, super-saturation translates into an increase in the lateral packing density of the molecules beyond a critical density point. The Langmuir technique is used to organize the molecules at an air-aqueous interface and subsequently compress them in two-dimensions to and beyond a critical density point. Examples of appropriate pressures include the pressures below the critical point for two-dimensional ordered structures and pressures at and above the critical density point for three-dimensional ordered structures. For example, in an embodiment, the population of amphiphilic molecules are compressed towards and beyond a critical density point, forming two different groups of protein domains. Large (200-500 μm) ordered structures are observed at intermediate packing densities, below the critical point. At higher packing densities, beyond the critical density point, formation of smaller (20-50 μm) ordered structures with a typical appearance of two-dimensional; or three dimensional crystal, may be observed using appropriate techniques, e.g., fluorescence. Preferably, the pressure is applied laterally (e.g., essentially parallel to the plane of the interface) such that a ordered structure is formed.

The term “interface” includes interfaces at which amphiphilic molecules can be applied, such that an ordered structure is formed. Examples of interfaces include gas-liquid, gas-solid, liquid-gel, liquid-liquid, liquid-solid, etc. In a further embodiment, the interface is gas-aqueous. Examples of gases that may be used include those which do not adversely affect the formation of the ordered structures. Some examples include gases such as argon, nitrogen, air, carbon dioxide, oxygen, etc. Examples of liquids that may be used include aqueous solutions (e.g., buffer solutions, water, saline solutions, glycerin solutions), organic solvents, or any other liquids which do not adversely affect the amphiphilic molecules or ordered structure.

The amphiphilic molecule may be contacted with the interface by any method which allows for the formation of the ordered structures of the invention. Preferably, a population of amphiphilic molecules retain their native asymmetry or a uniform orientation. For protein amphiphilic molecules, the proteins may be contacted with the interface in the presence of a lipid membrane. For example, the proteins may be applied to the interface from intact cells, or from a native cellular membrane of a cell where the protein was expressed or overexpressed, with or without further purification. Protein also may be applied to the interface by reconstitution in a liposome, proteoliposomes, or in a detergent solution, or by any method which allows for the formation of an ordered structure using the methods of the invention. In a further embodiment when the amphiphilic molecule is a protein (e.g., a membrane protein), the amphiphilic molecule is applied to the interface in a preparation which is essentially free of detergents (e.g., comprise less than about 0.1 or less percent detergent).

The ordered structures of the invention, such as self-similar microstructural arrays (SSOMA), may be formed through automated control of the growth mechanism using instrumentation known in the art (e.g. Mojtabai, F. (1989) Thin Solid Films 178: 115-123). The instrumentation can be used to probe directly (with high spatial resolution) the interactions between the molecules almost every step in the formation process.

The ordered structures of the invention may be formed by compression planar membrane, which may be comprised of the amphiphilic molecules of interest on an interface, prior to the formation of the ordered structure.

The term “planar membrane” includes monolayers, bilayers, and other membranes which are formed at the interface. The term “planar membrane” may be a monolayer, or bilayer, which when compressed, allows for the formation of ordered structures of populations of amphiphilic molecules. For example, amphiphilic molecules such as membrane proteins may be applied to the interface in the presence of lipids. The lipids and the proteins then form a planar membrane which then is compressed to form the protein ordered structures of the invention.

The term “planar membrane compression” is a method of forming ordered structures comprising contacting a population of amphiphilic molecules with a interface, compressing said population to an appropriate pressure laterally, such that a two-dimensional ordered structure is formed. In a further embodiment, the method includes formation of a planar membrane comprising the population of amphiphilic molecules prior to formation of the ordered structure.

### EQUIVALENTS

Those skilled in the art will recognize, or be able to ascertain using no more than routine experimentation, many equivalents to the specific embodiments and methods described herein. Such equivalents are intended to be encompassed by the scope of the following claims. All patents, patent applications, and literature references cited herein are hereby expressly incorporated by reference.

1. A method for determining the size of an amphiphilic molecule, comprising:
obtaining an ordered structure of amphiphilic molecules;
calculating the fractal dimensions of said ordered structure;
analyzing the fractal dimensions, such that the size of said amphiphilic molecule is determined.

2. The method of claim 1, wherein said amphiphilic molecule is a protein.

3. The method of claim 2, wherein said protein is a membrane protein.

4. The method of claim 1, wherein said ordered structure is a self-similar ordered microstructured array.

5. The method of claim 1, wherein said ordered structure is formed through planar membrane compression.

6. A method for generating a three dimensional template of an amphiphilic molecule, comprising:
obtaining at least two ordered structures of said amphiphilic molecule;
obtaining the 2D contour map of said amphiphilic molecule from said ordered structures;
analyzing said 2D contour maps, such that a three dimensional template of said amphiphilic molecule is generated.

7. The method of claim 6, wherein the analysis of the 2D contour maps comprises calculating the fourier transformation of the images.

8. The method of claim 6, wherein said amphiphilic molecule is a protein.

9. The method of claim 8, wherein said protein is a membrane protein.

10. The method of claim 6, wherein said ordered structure is a self-similar ordered microstructured array.

11. A method for obtaining a three dimensional structure of a protein, comprising:
obtaining at least two ordered structures of said protein;
determining the 2D contour maps of said protein from said ordered structures;
analyzing said 2D contour maps to obtain a three dimensional template;
fitting the amino acid sequence of said protein into said three dimensional template, thereby obtaining a three dimensional structure of said protein.

12. The method of claim 11, wherein said protein is a membrane protein.

13. The method of claim 11, wherein said ordered structures are obtained by planar membrane compression.

14. The method of claim 11, wherein said analysis of said 2D contour maps comprises calculating the fourier transformation of the images.

15. The method of claim 11, wherein the fitting of said amino acid sequence of said protein into said three dimensional template is optimized.
